(19) United States
(12) Patent Application Publication
(10) Pub. No.: US 2014/0349862 A1
(43) Pub. Date: Nov. 27, 2014

(54) METHODS AND COMPOSITIONS FOR THERAPEUTIC DRUG MONITORING AND DOSING BY POINT OF CARE PHARMACOKINETIC PROFILING

(71) Applicant: Vuong Ngoc Trieu, Agora Hills, CA (US)

(72) Inventor: Vuong Ngoc Trieu, Agora Hills, CA (US)

(21) Appl. No.: 13/261,786

(22) PCT Filed: May 30, 2012
(86) PCT No.: PCT/US2012/003993

§ 371 Date: Jun. 5, 2014

Related U.S. Application Data
(60) Provisional application No. 61/491,268, filed on May 30, 2011.
(60) Provisional application No. 61/514,488, filed on Aug. 3, 2011.
(60) Provisional application No. 61/526,950, filed on Aug. 24, 2011.
(60) Provisional application No. 61/533,250, filed on Sep. 11, 2011.
(60) Provisional application No. 61/577,008, filed on Dec. 17, 2011.
(60) Provisional application No. 61/606,371, filed on Mar. 3, 2012.
(60) Provisional application No. 61/615,312, filed on Mar. 25, 2012.
(60) Provisional application No. 61/635,730, filed on Apr. 19, 2012.

(51) Int. Cl. G01N 33/94; G01N 33/53

(52) U.S. Cl. CPC G01N 33/94; G01N 33/5308
USPC 506/9; 436/34; 436/501; 435/7.1; 435/7.92; 435/7.93

(57) ABSTRACT

Disclosed are methods and kits for pharmacokinetic profiling employing point-of-care or point of service self-sampling and allowing for dosage adjustments based on the pharmacokinetic profiles.

### CROSS-REFERENCE TO RELATED APPLICATIONS

This US non-provisional application claims the benefit of PCT/US2012/039993 which claims the benefit of priority to U.S. Provisional Applications Nos. 61/491,268, filed May 30, 2011, 61/514,488, filed Aug. 3, 2011, 61/526,950, filed Aug. 24, 2011, 61/533,250, filed Sep. 11, 2011, 61/577,008, filed Jan. 3, 2012, 61/606,371, filed Mar. 3, 2012, 61/615,312, filed Mar. 25, 2012, and 61/635,730, filed Apr. 19, 2012, each of which is incorporated by reference in its entirety.

### BACKGROUND OF THE INVENTION

Personalized medicine as it applies to tailoring treatment of an individual patient with therapeutic agents has a pharmacokinetics (PK) component where drug dose is optimized to the patient and a pharmacodynamics (PD) component where the treatment modality is matched to the patient. The treatment modality requires the understanding of the mechanism of action and mechanism of resistance. The identification of PD biomarkers has been the focus of considerable research effort, as evidenced by the tremendous growth of prognostic, predictive, and/or diagnostic biomarkers. However, the PK component is often neglected. Incomplete understanding of PK is now hampering PD biomarker testing from reaching its full potential.

Therapeutic drug monitoring (TDM) has the potential to revolutionize therapeutics by improving delivery of the drug as well as allowing for the correct application of biomarker testing. It is widely believed that identification of a biomarker predictive of response would allow targeted treatment of only those who would respond and spare those who would not respond to treatment. However, no matter how sensitive a disease is to a drug, an individual with the disease will not respond if the individual does not receive enough of the drug. This is where TDM would be expected to complement and advance the current effort on biomarker discovery.

However, personalized dosing has been typically a hit or miss process. Physicians are only given a general guideline as to the dosing of the patients, with much of the dose adjustment left to subjective observations of the patients. The Physician's Desk Reference summarizes experimentally-determined reasonable drug dosage ranges found in the research literature. These ranges are broad and the same dose is specified for all patients. Scientific research and publications exploring dose adjustment are not geared toward matching dose to individual patients. Rather, they provide a broad range of dosages based on averaging of characteristics over an entire population at worst, or only a subset of patients at best.

Researches recognize the need for finding new methods of accounting for inter-individual differences in drug response. Research over the past few decades has identified numerous factors that influence the clinical effects of medication. Age, gender, ethnicity, weight, obesity, concomitant morbidity, diet, and drug-drug interaction have all been found to influence both the pharmacokinetics and pharmacodynamics of drugs. The pediatric population, women, racial minorities, and the elderly often require different dosing schedule than their male Caucasian counterparts.

Because of the large number of potentially interacting variables affecting individual response to treatment, a physician faced with the task of minimizing side effects and maximizing drug performance currently must rely on trial and error to refine dosages prescribed for a given individual. Subjective and objective methods are used to identity adverse symptoms and to implement changes necessary during the course of treatment. A common method of monitoring is clinical observation, which involves individual counseling and close personal supervision by physicians, who observe physiological signs and symptoms of the disease. This method is error-prone, time consuming, expensive, highly subjective, and unduly increases the doctor-patient contact time. Furthermore, patients are at risk for unnecessary toxicity from exposure to high levels of drug, or for receiving suboptimal or ineffective levels of treatment from insufficient levels of drug.

Accordingly, there is a need for improved methods of dosing of individuals using by therapeutic drug monitoring.

### SUMMARY OF THE INVENTION

In certain embodiments, the present invention provides a method for therapeutic drug monitoring of an individual treated with a drug. The method involves constructing a pharmacokinetic profile of the drug for the individual using concentrations of drug in at least two samples obtained from the individual at time points suitable to construct a pharmacokinetic profile. The samples are collected at point-of-care or point of use by sampling or self-sampling on point-of-care devices or point of use devices, each capable of quantitating the drug, or on matrices suitable for storage of the at least two samples prior to quantitation of the drug by a laboratory. The pharmacokinetic profile includes pharmacokinetic parameters suitable for guiding dosing of the drug for the individual.

The samples are collected by at point-of-care or point of service, e.g., by self-sampling. Samples may be applied to a lateral flow device for quantitation of the drug, and the results transmitted to the physician or physician's agent for pharmacokinetic analysis. In other embodiments, the samples are collected at point-of-care or point of service, e.g., by self-sampling, on a suitable storage matrix, e.g., filter paper, prior to delivery of the samples to a laboratory for quantitation and analysis.

In certain embodiments, samples collected at various times from the individual through point-of-care or point-of-use by self-sampling are obtained by a laboratory. The laboratory then tests the samples to quantitate the drug of interest and, based on the results, constructs a pharmacokinetic profile. The results of the pharmacokinetic profile may be presented, optionally along with recommendations to increase the individual's dosage of the drug in order to enhance efficacy or to reduce the dosage in order to reduce risk of toxicity.

In another aspect it provides a kit for therapeutic drug monitoring of an individual treated with a drug using pharmacokinetic profiling. Advantageously, the kit may be used to perform the method of claim 1. The kit comprises a plurality of point-of-care device or a point of use device capable of quantitating the drug in the at least two samples, or matrices suitable for storage of the samples prior to quantitation by a laboratory.

### BRIEF DESCRIPTION OF THE DRAWINGS

FIG. 1 is a graph showing a PK profile from bolus intravenous administration of a drug and a PK profile from oral administration of a drug.

FIG. 2 is a graph showing AUC as a function of dose for Taxol administered over 3 hours or 24 hours.

FIG. 3 is a graph showing AUC as a function of dose for two different formulations of paclitaxel.

FIG. 4 is a graph showing AUC as a function of dose for two different formulations of paclitaxel.

FIG. 5 is a graph showing AUC as a function of dose for two different formulations of paclitaxel.

FIG. 6 is a graph showing mean paclitaxel concentration as a function of time from 0 to 72 hours after the start of the infusion for two different formulations of paclitaxel.

FIG. 7 is a series of graphs presenting results of studies showing the variability of Abraxane pharmacokinetic parameters Vz (FIG. 7A), AUCinf (FIG. 7B), Cₘₐₓ (FIG. 7C), CL (FIG. 7D), and T_{1/2} (FIG. 7E) within a population receiving Abraxane.

FIG. 8 is a series of graphs presenting results of studies showing the variability of FSH pharmacokinetic parameters Cₘₐₓ (FIG. 8A), AUC (FIG. 8B), CL (FIG. 8C), and T_{1/2} (FIG. 8D) within a population receiving 150 IU FSH.

FIG. 9 is a series of graphs presenting results of studies showing the variability of FSH pharmacokinetic parameters Cₘₐₓ (FIG. 9A), AUC (FIG. 9B), CL (FIG. 9C), and T_{1/2} (FIG. 9D) within a population receiving 300 IU FSH.

FIG. 10 shows a profile of single exponential PK curve-assuming bolus administration.

FIG. 11 shows a PK profile characterized by two exponentials.

FIG. 12 shows a prototypic paclitaxel lateral flow assay

FIG. 13 shows the results of a quantitative lateral flow for a hCG using a confocal photometric scanner.

FIG. 14 FSH quantitative lateral flow using an image analysis program coupled with a camera.

FIG. 15 Stability of the signal for nanogold quantitative FSH lateral flow.

FIG. 16 Quantitative point-of-care lateral flow assay for LH-Luteinizing hormone. Urine LH Assay (range=5-1,700 IU/L); Serum/Plasma LH Assay (range=1-1,700 IU/L); Blood LH Assay (range=5-1,700 IU/L).

FIG. 17 Quantitative point-of-care assay for FSH-follicle stimulating hormone. Urine FSH Assay (range=6-1,500 IU/L); Serum/Plasma FSH Assay (range=5-10,000 IU/L); Blood FSH Assay (range=5-10,000 IU/L).

FIG. 18 Quantitative point-of-care assay for hCG—human chorionic gonadotropin. Urine hCG Assay (range=5-10,000 IU/L); Serum hCG Assay (range=1-10,000 IU/L); Blood hCG Assay (range=20-10,000 IU/L).

FIG. 19 is a summary of estimated paclitaxel blood pharmacokinetic variables for Abraxane and Taxol (pharmacokinetic population).

### DETAILED DESCRIPTION OF THE INVENTION

The present invention relates to methods by which pharmacokinetic profiles of drugs may be constructed for individuals receiving a drug using samples obtained at various time points following dosing. Data for use in the construction of the pharmacokinetic profiles is obtained from samples collected at point-of-care or point of use. Advantageously, the samples may be obtained by self-sampling. In certain embodiments, the samples may be delivered to a point-of-care device to quantitate the drug, and the results thus obtained are reported to the physician or his agent. Alternatively, the samples are collected using a matrix or vessel suitable for collection and storage of the samples until receipt and analysis by a laboratory. Examples of matrices suitable for collection and storage of the samples include, but are not limited to commercially available biological sampling filter paper systems such as Whatman 3 MM, GF/CM30, GF/QA30, S&S 903, GB002, GB003, or GB004. Several categories of blotting materials for blood specimen collection are available, e.g., S&S 903 cellulose (wood or cotton derived) filter paper and Whatman glass fiber filter paper. The blood spot is placed in one or more designated areas of the filter paper, allowed to dry, and then mailed along with a test request form to the laboratory. This method of collection has the advantage of obviating the need for collection of samples at a doctor's office or clinic. Thus, multiple samples may be conveniently collected by the patient over a period of from 0 to 72 hours at considerable savings of cost and time. This has the advantages of increased efficiency and reduced delays in transmitting results of the analysis to the treating physician, who may use the information to adjust treatment as necessary, and contact the patient to convey the new treatment regimen.

Prior to the instant invention, the potential value of pharmacokinetic-guided dosing had not been exploited in part because collecting the samples needed for individual pharmacokinetic profiles was inconvenient and prohibitively expensive, in that collection of samples typically requires extended hospital stays of up to 72 hours. For a typical drug PK study, pharmacokinetic studies have been limited to phase I studies in which PK/PD on a few patients forms the basis for the use of the drug throughout the population. To compensate, a population PK study is usually performed during phase III of the drug development; however, due to the same limitations, these studies are performed under the sparse sampling procedure. Due to the imprecision associated with sparse sampling, only an approximation of population PK can be obtained. For full pharmacokinetic testing, at least 12 data points collected over a period of 48-72 hours are needed to adequately characterize the PK parameters for each particular patient. It is not possible to perform this for every patient enrolled in a phase Ill (or even for a phase II trial for that matter). Obtaining blood samples to assess drug levels immediately after drug administration for Cₘₐₓ are not typically problematic. However, obtaining blood samples at subsequent time points to define “Clearance” and “Concentration at Steady State” are difficult as the inflection points are 4-24 hours after treatment. It is often inconvenient or burdensome for patients to return to the hospital for more blood work, and keeping the patient overnight would be expensive both in terms of cost and services.

A phase 3, multicenter, randomized study (N=208) in which 5-FU dosing was optimized at steady state levels by LC/MS demonstrates the benefits of pharmacokinetic-guided dosing for both efficacy and toxicity (Gamelin, E, Delva, R, Jacob, J, et al: “Individual fluorouracil dose adjustment based on pharmacokinetic follow-up compared with conventional dosage: Results of a multicenter randomized trial of patients with metastatic colorectal cancer.” J Clin Oncol. 13:2099-2105, 2008.). Half of the patients were dosed with 5-FU based on body surface area (BSA). The other half were initially dosed based on BSA, with subsequent cycle doses adjusted based on blood tests that measured the actual concentration of chemotherapy in the patients' blood plasma. The primary endpoint was tumor response; the secondary endpoint was treatment tolerance. The study concluded that: 1) Response rates were nearly doubled in the dose-adjusted group versus the BSA group (33.6 percent versus 18.3 percent) with statistical significance; 2) Overall survival at two years among patients with personalized 5-FU dose management improved by 48 percent with an improved median survival of 22 months versus 16 months in the BSA arm. The survival data was leaning towards significance; 3) Grade III/IV 5-FU related toxicities were found to be significantly lower in patients with personalized dose adjustment; 4) Fifty eight percent of patients were found to be under-dosed (sub-therapeutic and less effective drug levels) and had their doses adjusted upwards; and 5) Seventeen percent were found to be over-dosed (increasing the risk of severe side effects) and had their doses adjusted downward. However, most intravenously administered drugs are not dosed to achieve steady state with prolonged infusion of 24 hours Instead, they are dosed as single short infusions (30 min-3 hours) not allowing for long sustained steady state drug concentrations in the blood.

Drug PK is characterized by the uptake of the drug into various compartments of the body—generally traversing the blood, tissues, and then the target organ. It is also characterized by the clearance of the drug from body by either active metabolism of the drug, or clearance by the liver and/or the kidney. The blood concentration versus time curve describes how the drug is being handled by the individual. The commonly accepted parameter describing drug exposure is area under the curve (AUC) or time over effective drug concentration or time over toxic drug concentration. It is generally accepted that there is a single universal PK profile for each drug. However, this is not true as survey of the PK literature would reveal wide variation in PK profiles for any particular drug. This is probably due to individual variability in uptake and clearance. In fact, two completely different profiles can have similar AUC values. Therefore, to fully exploit TDM-guided dosing, a fully personalized PK profile will have to be generated for each patient so that dose optimization can be performed and dose adjustment by changes in frequency or duration of dosing can be estimated using conventional PK programs such as Phoenix (WinNonlin). Using any one single PK parameter for dose adjustment would be ignoring the wealth of PK data available and would not be as effective. Therefore, a fully personalized PK profile would be useful in guiding dose adjustment, and would allow for more accurate dose adjustments than a single PK parameter such as AUC.

In certain embodiments, a solution to this problem is the development of a lateral flow test that can be performed using blood from a single finger prick. Ideally, the test involves application of a single prick to a lateral flow device, which in turn gives a readout that is sent to the attending physician via Wifi/Internet. In certain embodiments, meaningful PK data may be obtained by patients testing themselves for at least 2, 3, 4, 5, 6, 7, 8, 9, 10, 11, or 12—specified time points across a period of from 0-24 hours, 0-36 hours, 0-48, or 0-72 hours

There is a need for improved assays that can be used to more readily detect small molecule analytes, and to detect low concentrations of analyte. In addition, there is a need for improved measurement of analytes including small molecule analytes in order to customize drug regimens to maintain efficacy of the drug while reducing unwanted side effects in individual patients. Furthermore, there is a need for methods and apparatuses that can be used at the point-of-care to measure biologically and/or clinically relevant analytes in order to reduce the delay between obtaining the sample and obtaining the results of the assay.

The drugs to be monitored by the invention described herein can be any specific substance or component that one is desirous of detecting and/or measuring in a chemical, physical, enzymatic, or optical analysis. Such analytes include, but are not limited to, therapeutic and/or pharmacologic agents (e.g., theophylline, anti-HIV drugs, lithium, anti-epileptic drugs, cyclosporin, chemotherapeutics). In preferred embodiments, the analyte is a physiological analyte of interest or a chemical that has a physiological action, for example a drug or pharmacological agent. The terms drugs and pharmacological agents are used interchangeably and may include any molecule having a therapeutic effect, including small molecules and biologics.

In certain embodiments, the drugs to be monitored may include but are not limited to chemotherapies, fertility treatment agents, anti-viral agents, and anti-microbial agents.

In certain preferred embodiments, the methods of the invention are used to monitor patients who would benefit from dose adjustment due to the PK variability of the specific therapeutics they are receiving.

Chemotherapeutic agents that may be monitored using the methods of the invention include but are not limited to paclitaxel, docetaxel, gemcitabine, and 5-fluorouracil. The methods may be used to monitor various formulations of paclitaxel, including, for example, Abraxane, Taxol, or Genexol, or Nanoxel, or Zyotag, or Tocosol, or other formulations. These include but are not limited to albumin-bound paclitaxel, polymeric micelle paclitaxel, polymeric nanoparticle formulation of paclitaxel, vitamin E-based paclitaxel emulsion, polyglutamate paclitaxel, as well as formulations currently in development or yet to be developed, such as lipid dispersion paclitaxel, targeted paclitaxel nanoparticles (albumin based as well as PEG-based), and porous paclitaxel nanoparticles.

Fertility treatment agents that may be monitored include, for example, examples are FSH, LH, hCG, GnRH agonists/antagonists, estradiol and others

Anti-Viral/Microbial agents that may be monitored include without limitation protease inhibitors, reverse transcriptase inhibitors such as 2′,3′-dideoxyinosine (ddl), 2′,3′-dideoxycytidine (ddC) or 3′-azido-2,3′-dideoxythymidine (AZT), anti-fungal drugs, e.g. itraconazole which is also used in anti-leishmanial chemotherapy, or antimonials; the most used anti-leishmanial drug.

Additionally, patients receiving certain classes of therapeutics would benefit from dose adjustment due to PK variability. Examples of these classes of therapeutic include oral agents, intravenous agents, and Intramuscular/Intravaginal/intraorbital agents.

The PK profiles for oral agents are generally characterized by slow absorption followed by steady decline in blood concentration. Tₘₐₓ and Cₘₐₓ are important parameters that describe the PK profile as well as bioavailability and steady state level. These include antiviral agents which are often orally administered, as well as targeted therapies for cancer, anti-hypercholesterolemic agents, anti-hypertensive agents, etc.

For intravenous agents, including small molecules and biologics, Cₘₐₓ is important as definition of toxicity as well as AUC. These include most chemotherapeutic agents and Antibody Drug Conjugates (ADCs) used in cancer therapies. The PK profile for this class is characterized by rapid increase in Cₘₐₓ that reaches steady state during the infusion and then a rapid drop following infusion stop and slow decay thereafter.

For agents administered by intramuscular/intravaginal/intraorbital routes, including biologics and small molecules, the steady state concentration, absorption, terminal T_{1/2} are important parameters. This class of drugs includes the hormones used in fertility treatment and other biologics such as Etanercept, Rituximab, rosuvastatin, trastuzumab, insulin, factor VIII, tPA, up, antibody-drug conjugates, etc. These agents exhibit PK profiles that are an intermediate between oral agents and intravenous agents. Therapeutic drugs include, for example, immunosuppressant medications such as cyclosporin, tacrolimus (FK-506), rapamycin, and mycophenolic mofetil. Immunosuppressants have a narrow therapeutic index and high degree of inter- and intra-patient variability in bioavailability. Immunosuppressants require careful, frequent monitoring in patient samples in order to balance the level of immunosuppression needed to prevent transplant rejection while avoiding the adverse effects of excessive immunosuppression such as toxicity, infection, and increased risk of developing cancer. These include antiviral agents such as anti-HIV. Examples are anti-retroviral drugs, such as proteinase or reverse transcriptase inhibitors, e.g. 2′,3′-dideoxyinosine (ddl), 2′,3′-dideoxycytidine (ddC) or 3′-azido-2,3′-dideoxythymidine (AZT)

Therapeutic drug monitoring is predicted to be most effective against drugs with highly variable PK such as paclitaxel. Paclitaxel has more than ten-fold variability in patient exposure (Evans, W. E. & Relling, M. V. Clinical pharmacokinetics-pharmacodynamics of anticancer drugs. Clin. Pharmacokinet. 16, 327-336 (1989); Freyer, G. et al. Pharmacokinetic studies in cancer chemotherapy: usefulness in clinical practice. Cancer Treat. Rev. 23, 153-169 (1997); Masson, E. & Zamboni, W. C. Pharmacokinetic optimisation of cancer chemotherapy: effect on outcomes. Clin. Pharmacokinet. 32, 324-343 (1997). Various patient-related factors can affect drug pharmacokinetics (PK), for example organ function, expression and activity of metabolizing enzymes, drug resistance, body size, gender, age, concomitant disease and co-administration of other drugs. These factors may be of clinical significance in chemotherapy dose determination. Paclitaxel is eliminated by the liver into feces (Sparreboom A, Scripture C D, Trieu V, Williams P J, De T, Yang A, Beals B, Figg W D, Hawkins M, Desai N. Comparative preclinical and clinical pharmacokinetics of a cremophor-free, nanoparticle albumin-bound paclitaxel (ABI-007) and paclitaxel formulated in Cremophor (Taxol). Clin Cancer Res. 11, 4136-43 (2005); Sparreboom A, van Tellingen O, Nooijen W J, Beijnen J H. Tissue distribution, metabolism and excretion of paclitaxel in mice. Anticancer Drugs. 7, 78-86 (1996)); therefore, hepatic impairment impacts on the clearance of drug from circulation and Permeability glycoprotein (Pgp), also known as multidrug resistance protein 1 (MDR1), status impacts on the clearance of drug and resorption of in the gut. Hepatic clearance is further influenced by CYP factors (Walle T. Assays of CYP2C8- and CYP3A4-mediated metabolism of taxol in vivo and in vitro. Methods Enzymol, 272, 145-51 (1996)). Additionally, there are other factors which are inherent to the use of body surface area (BSA) as dose normalizer (Baker, S. D. et al. Role of body surface area in dosing of investigational anticancer agents in adults, 1991-2001. J. Natl. Cancer Inst. 94, 1883-1888 (2002). BSA itself is affected by weight; paclitaxel AUC increases with increasing weight due to increase in total dose in heavier patients. As a consequence, Caucasians tend to have higher AUC than Asians. The effect of age—as most patients are elderly—has not been adequately analyzed.

For paclitaxel, there exists a large inter-individual variation in pharmacokinetics; for AUCinf the average CV is in the range of 20-50% (Sparreboom A, Scripture CD, Trieu V, Williams P J, De T, Yang A, Beals B, Figg W D, Hawkins M, Desai N. Comparative preclinical and clinical pharmacokinetics of a cremophor-free, nanoparticle albumin-bound paclitaxel (ABI-007) and paclitaxel formulated in Cremophor (Taxol). Clin Cancer Res. 11, 4136-43 (2005); Nyman D W, Campbell K J, Hersh E, Long K, Richardson K, Trieu V, Desai N, Hawkins M J, Von Hoff D D. Phase I and pharmacokinetics trial of ABI-007, a novel nanoparticle formulation of paclitaxel in patients with advanced nonhematologic malignancies. J Clin Oncol. 23, 7785-93 (2005); Yamada K, Yamamoto N, Yamada Y, Mukohara T, Minami H, Tamura T. Phase I and pharmacokinetic study of ABI-007, albumin-bound paclitaxel, administered every 3 weeks in Japanese patients with solid tumors. Jpn J Clin Oncol. 40:404-11 (2010); Gardner E R, Dahut W L, Scripture C D, Jones J, Aragon-Ching J B, Desai N, Hawkins M J, Sparreboom A, Figg W D. Randomized crossover pharmacokinetic study of solvent-based paclitaxel and nab-paclitaxel. Clin Cancer Res. 14: 4200-5 (2008); Stinchcombe T E, Socinski M A, Walko C M, O'Neil B H, Collichio F A, Ivanova A, Mu H, Hawkins M J, Goldberg R M, Lindley C, Claire Dees E. Phase I and pharmacokinetic trial of carboplatin and albumin-bound paclitaxel, ABI-007 (Abraxane) on three treatment schedules in patients with solid tumors. Cancer ChemotherPharmacol. 60:759-66 (2007). The cause of this large variation is not completely understood and therefore cannot be controlled for. Hepatic impairment and obesity have been identified as contributing factors; however, other unknown factors remain to be defined. Looking at paclitaxel PK, it is apparent that patients dosed with the approved dose, have a high chance of being either underdosed or overdosed (Mielke S. Individualized pharmacotherapy with paclitaxel. Curr Opin Oncol. 19, 586-9 (2007).

The highly variable paclitaxel PK does have a significant impact on its therapeutic application. In two studies where paclitaxel dose of 100 mg/m² was compared to paclitaxel dose of 125 mg/m², the higher dose group did better than the lower dosed group (Blum J L, Savin M A, Edelman G, Pippen J E, Robert N J, Geister B V, Kirby R L, Clawson A, O'Shaughnessy J A. Phase II study of weekly albumin-bound paclitaxel for patients with metastatic breast cancer heavily pretreated with taxanes. Clin Breast Cancer 7:850-6 (2007); Von Hoff D D, Ramanathan R K, Borad M J, Laheru D A, Smith L S, Wood T E, Korn R L, Desai N, Trieu V, Iglesias J L, Zhang H, Soon-Shiong P, Shi T, Rajeshkumar N V, Maitra A, Hidalgo M. Gemcitabine Plus nab-Paclitaxel Is an Active Regimen in Patients With Advanced Pancreatic Cancer: A Phase I/II Trial. J ClinOncol. 29:4548-54 (2011). Given the high variability of paclitaxel PK, it is highly probable that identification of patients who are underdosed and moving them to higher dose would result in more than a 25% increase in drug exposure. In view of the foregoing, TDM of paclitaxel is likely to be beneficial.

The methods of the invention would have applicability in monitoring diseases that are characterized by a relatively high frequency of resistance to treatment such as cancers and HIV. Treatment of these conditions would be enhanced by frequent monitoring of drug levels in conjunction with resistance testing. Therefore, methods have been developed which monitor the phenotypic alterations in the population of HIV virions circulating in the patient (e.g. WO 97/27480). Other phenotyping assays include those described by Witvrouw (WO 01/57245), Virologic (WO97/27319) and Bioalliance (WO 02/38792). The Antivirogram® (WO97/27480) estimates the drug susceptibility of the viral population, as compared to ‘wild-type’ strains. In this test service, the drug concentration that inhibits virus growth for 50% (IC50) is determined in vitro. The ratio IC50 of the virus in a patient's blood sample over the IC50 of “wild-type” virus is the fold change in drug susceptibility of the virus in that patient.

The minimum plasma concentration or trough concentration can be critical during treatment of diseases or conditions wherein the drug target is subject to modulation in order to overcome the drug effect. Examples of this latter phenomenon are found in diseases like viral infections such as HIV, bacterial infections, and cancer. For example, the presence of drugs generates mutational pressure on the HIV protease to escape from drug therapy and insufficient inhibitor concentration facilitates such escape. The determination of the drug levels in a patient and the use of this value to determine the trough level can be important in obtaining dosages high enough to yield a therapeutic effective concentration.

One aspect of the methods of the instant invention is the determination of pharmacological parameters of the drug in an individual, including trough levels (Ct), maximal concentration (Cₘₐₓ), the area under the curve (AUC), elimination velocity, etc. These parameters may be inputted in a population pharmacokinetic model and pharmacokinetic variables such as Ct, Cₘₐₓ, and AUC may be calculated (WO 02/23186). These pharmacological variables may be used, for example, to estimate the potential toxicity of a certain dose, exposure time to a drug (e.g. in case of radiochemicals), or minimum concentration.

In order to obtain effective treatment, the exposure of an individual to a drug as determined, for example, by trough level or AUC, must exceed a certain level. This level is determined by the nature of the virus population. The ratio of the exposure to the drug (trough level, AUC, other) over the drug-resistance (fold-resistance, IC50, IC90, etc.) is predictive for successfulness of the therapy. This ratio can be expressed as the IQ (inhibitory quotient) (IQ=Ct/IC50). This IQ value can be further normalized in order to obtain a value adjusted for protein binding (normalized IQ, NIQ). One approach of obtaining this adjusted value is to determine the mean Ct for a population, and the IC50 for an active ingredient for a reference strain i.e. a reference laboratory HIV strain. The quotient of these latter two values yields (Ct/IC50) reference. The normalized IQ is provided by the quotient: [(Ct/IC50)] patient/[(Ct/IC50)] reference.

According to the present invention a “biological sample” includes any sample derived from an organism, i.e. a human or animal, optionally comprising an active ingredient. A biological sample includes but is not limited to blood, serum, plasma, saliva, cerebrospinal fluid, ejaculate, mammary ductal lavage and hair. In certain preferred embodiments, the sample is blood. A biological sample further includes samples obtained from culture flasks, wells, and other types of containers. The biological sample may comprise one or more active ingredients.

An active ingredient may include any compound, including a chemical, drug, antibody, ligand, antisense compound, aptamer, ribozyme, peptide, non-natural peptide, protein, PNA (peptide nucleic acid) and nucleic acids or a composition including at least one compound. Active ingredient further comprises the compounds as administered and their metabolites. Metabolites may be generated under physiological conditions. As used in the present invention the level of an active ingredient means the amount or concentration of said active ingredient in said sample. The active ingredient present in the biological sample may differ from the active ingredient in the reference. Under these circumstances, the inhibitory potency of the biological sample equals an amount or concentration of active ingredient derived from a reference standard curve. The amount can be expressed as for example, g, ml, mol. The concentration can be expressed for example as ml/ml, g/l, M and the like.

Pharmacokinetic profiles constructed using at least two datapoints will be used to generate PK parameters that can be used to guide dose adjustment. The parameters include: AUCinf, Tₘₐₓ, Cₘₐₓ, Time above threshold, T_{1/2}, V_{d}, Vz, Tₘₐₓ, lambda, exponentials, tau. etc. These include parameters from noncompartmental PK analyses and compartmental PK analyses (physiological based or not). These PK parameters are derivatives of the actual PK profile and one or more may be needed to adequately allow for dose adjustment. The determination of which parameters or how many, are to be determined pragmatically during clinical development of the drug. Based on the PK profile for an individual, the dose may be adjusted to reduce toxicity and/or enhance efficacy of a given drug. For example, neutropenia associated with paclitaxel treatment is related to time above 50 nM of paclitaxel. Therefore, this parameter could be used to guide dose adjustment to reduce the risk of that individual developing neutropenia. Similarly, measures of drug exposure such as AUC can be used to guide dose adjustment to enhance efficacy. The complexity of the PK parameters and PK profile will be determined following clinical trial comparing response to PK profile and its parameters. Using multivariate analysis, logistic regression, neural net and other suitable statistical analyses, the appropriate defining adjustment parameters will be derived to facilitate dose adjustment.

Dose adjustment can be made to within the expected median of the particular drug. For example, the dosage may be adjusted to achieve a level that is within 5%, 10%, 15%, or 20% of the desired target value. In cases where the effective dose of the drug is known and is also variable for the patient, dose adjustment will be performed as therapeutic quotient to maintain sufficiently high therapeutic effectiveness without approaching toxicity. In some cases, this may not be possible.

The use of the pharmacokinetic profiles to guide dosing would decrease the unpredictability of dosing, increase efficacy, and decrease toxicity by increasing the dose for underdosed patients and decreasing the dose of overdosed patients. Underdosed and overdosed patients are defined by the PK profile and its associated parameters.

Non-limiting examples of suitable devices or methods of testing drugs include lateral flow devices for the determination of the concentration of an analyte in a sample comprising providing a lateral flow strip for use in measuring the analyte. Examples of analytes that may be tested include therapeutic drugs, drug metabolites, and hormones. Application of the sample to the lateral flow strip causes a fraction of the analyte in the sample to bind to a component of the lateral flow strip such that a detectable signal proportional to the concentration of the analyte in the sample is produced.

Alternatively, the quantitation may be conducted on samples submitted by individuals to a laboratory by any suitable assay, including, but not limited to, those currently known to the art, such as ELISA, liquid chromatography-mass spectrometry (LC-MS), thin layer chromatography (TLC), high-performance liquid chromatography (HPLC), and mass spectrometry (MS) or other traditional assays for drug monitoring at central lab have been well illustrated. The samples could be whole blood collected following a finger prick on a suitable matrix and stored as a dry blood spot that is shipped or otherwise delivered to a laboratory for testing. Sampling can be performed with capillary and/or device designed to deliver a precise and small amount of blood to the dried blood spot card.

### EXAMPLES

### Example 1

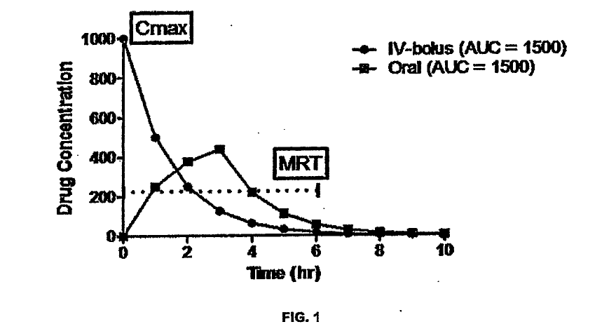

Theoretical PK profiles are shown in FIG. 1. As shown in FIG. 1, the PK profile has two principal components—the uptake portion of the curve immediately after administration and the decay/clearance part of the curve after maximum blood concentration (Cₘₐₓ). This theme varied from immediate Cₘₐₓ with bolus administration to delayed Cₘₐₓ with oral administration. In between are intravenous infusion, intranasal, intrabuccal, intramuscular, intraperitoneal, etc. The three main characteristics of PK profile are: Cₘₐₓ (maximum blood concentration), Mean Residence Time (MRT), and AUC (Area Under Curve). AUC is commonly used to indicate blood exposure. However, two curves with similar AUCs, as depicted in FIG. 1, can have very different profiles. The intravenous bolus dosing exhibits high initial blood concentration as define by Cₘₐₓ=Dose/blood volume. The oral dosing exhibits long residence time of the drug due to delayed absorption. Pharmaceutical development normally relies on experimentation to define how to best deliver the drug for maximum activity and minimum toxicity. It is surprising that therapeutic drug monitoring (TDM) is only relying on single PK parameter or single determination of blood concentration. It is this invention that suggests totality of PK data (PK profiling) should be used for therapeutic drug monitoring, as factors affecting either absorption of the drug or clearance of the drug are personal characteristics that cannot yet be predicted. The direct determination of personal PK profile should be a tool for personalizing dosing.

### Example 2

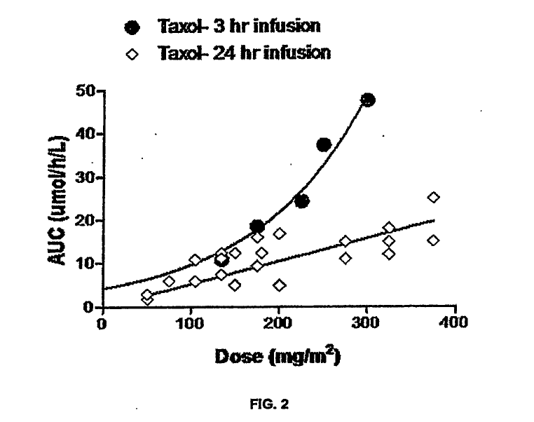

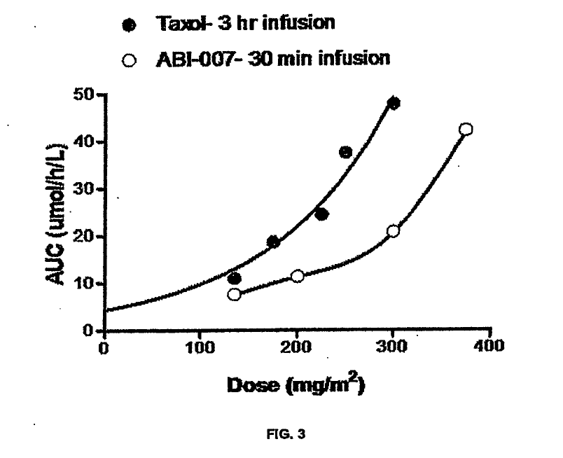

To demonstrate that simply changing the intake of drug would change the PK profile, we examined PK profile of Taxol dosed at 3 hr. infusion rate and a slower 24 hr. infusion rate using published pharmacokinetic data for Taxol (Ohtsu T, Sasaki Y, Tamura T, Miyata Y, Nakanomyo H, Nishiwaki Y, Saijo N. (1995) Clinical pharmacokinetics and pharmacodynamics of paclitaxel: a 3-hour infusion versus a 24-hour infusion. Clin Cancer Res. 1:599-606.; Wiemik P H, Schwartz E L, Einzig A, Strauman J J, Lipton R B, Dutcher J R (1987) Phase I trial of taxol given as a 24-hour infusion every 21 days: responses observed in metastatic melanoma. J Clin Oncol. 5:1232-9.; Tamura T, Sasaki Y, Eguchi K, Shinkai T, Ohe Y, Nishio M, Kunikane H, Arioka H, Karato A, Omatsu H, et al. (1994) Phase I and pharmacokinetic study of paclitaxel by 24-hour intravenous infusion. Jpn J Cancer Res. 85:1057-62.; Tamura T, Sasaki Y, Nishiwaki Y, Saijo N. (1995) Phase I study of paclitaxel by three-hour infusion: hypotension just after infusion is one of the major dose-limiting toxicities. Jpn J Cancer Res. 86:1203-9.) Paclitaxel dosed at the same doses, with a simple variation of infusion time, gave completely different AUCs (FIG. 2). The 3 hr infusion gave higher AUCs or drug exposure than the 24 hr infusion. The variation in uptake efficiency—which would affect the curve the same way as shortening of infusion time—is showing a strong impact on drug exposure as defined by PK profiling. This reinforces the concept that PK profile is highly variable and subjected to parameters known to vary among patients. These include absorption efficiency, rate of distribution out of blood and into tissues, and protein binding characteristics, and others. A single factor such as AUC cannot define drug exposure.

### Example 3

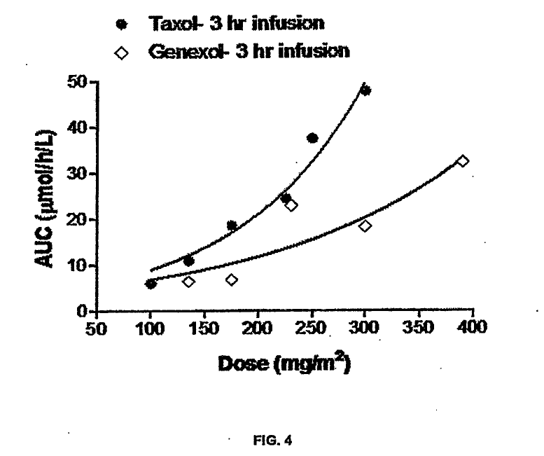

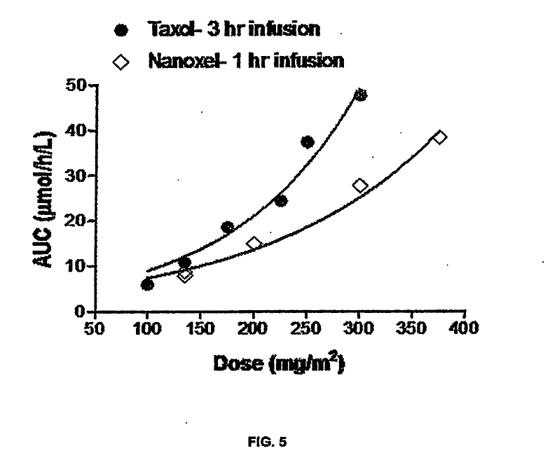

To demonstrate that an increase in tissue distribution rate and therefore a decrease in the rate of drug accumulation in blood would alter the PK profile, we examined the PK profile of Abraxane versus Taxol. As shown in FIG. 3, paclitaxel formulated as Abraxane (ABI-007) exhibited lower AUC than paclitaxel formulated as Taxol (Cremophor EL). The increase in AUC with faster uptake/infusion (30 min infusion for Abraxane versus 3 hr infusion for Taxol) was not observed. Abraxane® (albumin-bound paclitaxel, Abraxis BioSciences)—FDA approval in 2005 for metastatic breast cancer. It is known that Abraxane formulation resulted in faster tissue penetration—a property that is known to varied from individual to individual dependent on their level of paclitaxel binding proteins and others. This again exemplified that PK profiling—the use of totality of PK data—is more suitable for therapeutic drug monitoring. We found the same with other Cremophor free formulation such as Genexol (FIG. 4) and Nanoxel (FIG. 5). Genexol-PM® (Methoxy-PEG-poly(D,L-lactide) taxol; Samyang, Korea)—approved in S. Korea for metastatic breast cancer with Phase II for pancreatic cancer in the US.

### Example 4

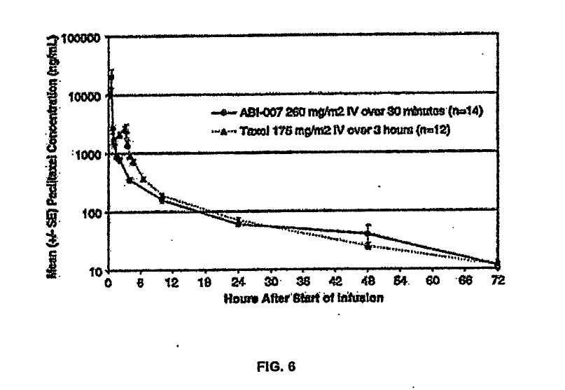

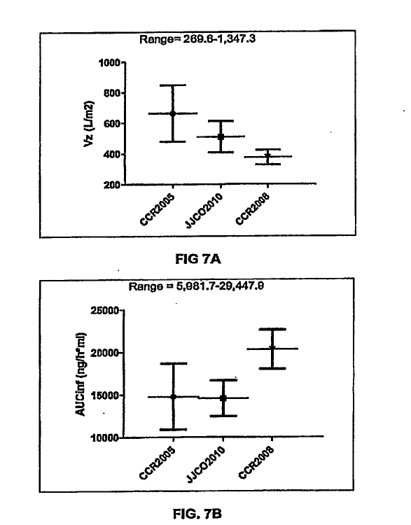

A comparison of the PK profiles of Abraxane (ABI-007) and Taxol clearly demonstrates the need for PK profiling to predict response and adjust dosages. As stated above, Abraxane PK profile is different from Taxol PK profile, with AUC being higher for Taxol at same doses. FIG. 6 shows the mean paclitaxel concentrations versus time from 0 to 72 hours after the start of the infusion of Abraxane or Taxol. When Abraxane was dosed at 260 mg/m² and Taxol was dosed at 175 mg/m², they exhibited mostly the same PK parameters (Table I). The PK profile for Abraxane and Taxol were very similar at this dose level (Sparreboom A, Scripture C D, Trieu V, Williams P J, De T, Yang A, Beals B, Figg W D, Hawkins M, Desai N. (2005) Comparative preclinical and clinical pharmacokinetics of a cremophor-free, nanoparticle albumin-bound paclitaxel (ABI-007) and paclitaxel formulated in Cremophor (Taxol). Clin Cancer Res. 11:4136-43.); and yet, ABI-007 demonstrated significantly higher response rates compared with standard paclitaxel (33% vs. 19%, respectively; P=0.001) and significantly longer time to tumor progression (23.0 vs. 16.9 weeks, respectively; hazard ratio=0.75; P=0.006) (Gradishar W J, Tjulandin S, Davidson N, Shaw H, Desai N, Bhar P, Hawkins M, O'Shaughnessy J. (2005) Phase III trial of nanoparticle albumin-bound paclitaxel compared with polyethylated castor oil-based paclitaxel in women with breast cancer. J Clin Oncol. 23:7794-803.). This is reinforcing that PK profile—or totality of PK data—need to be used to evaluate efficacy of a drug dose.

Table 1 (FIG. 19) is a summary of estimated paclitaxel blood pharmacokinetic variables for Abraxane and Taxol (pharmacokinetic population).

### Example 5

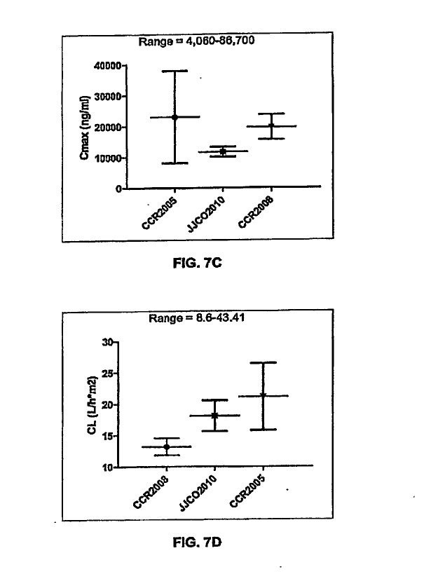

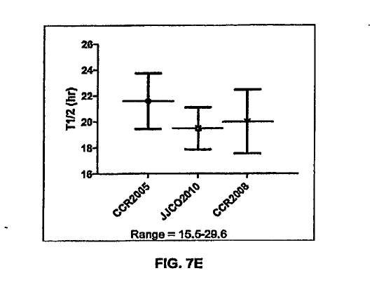

To determine whether Abraxane PK has enough variability to benefit from therapeutic drug monitoring, we performed a PK analysis using all published literature on Abraxane PK. Shown below are the plots of Abraxane PK across several studies (FIG. 7) (Yamada K, Yamamoto N, Yamada Y, Mukohara T, Minami H, Tamura T. (2010) Phase I and pharmacokinetic study of ABI-007, albumin-bound paclitaxel, administered every 3 weeks in Japanese patients with solid tumors. Jpn J Clin Oncol. 40:404-11.; Gardner E R, Dahut W L, Scripture C D, Jones J, Aragon-Ching J B, Desai N, Hawkins M J, Sparreboom A, Figg W D. (2008) Randomized crossover pharmacokinetic study of solvent-based paclitaxel and nab-paclitaxel. Clin Cancer Res. 14:4200-5; Nyman D W, Campbell K J, Hersh E, Long K, Richardson K, Trieu V, Desai N, Hawkins M J, Von Hoff D D. (2005) Phase I and pharmacokinetics trial of ABI-007, a novel nanoparticle formulation of paclitaxel in patients with advanced nonhematologic malignancies. J Clin Oncol. 23:7785-93.). The ranges for all variables were 209%, 142%, 458%, 200%, 69% of the mean for Vz, AUC, Cₘₐₓ, CL, and T_{1/2}, respectively. In contrast, we calculated that 5-FU AUC exhibits a range of 3.9-16.41 and mean of 9.05, or 138% variability (Ychou M, Duffour J, Pinguet F, Kramar A, Joulia J M, Topart D, Bressolle F. (1999) Individual 5-FU dose adaptation schedule using bimonthly pharmacokinetically-modulated LV5-FU2 regimen: a feasibility study in patients with advanced colorectal cancer. Anticancer Res. 19:2229-35.). Since 5-FU has been proven to benefit from therapeutic drug monitoring dose adjustment (Gamelin, E, Delva, R, Jacob, J, et al: “Individual fluorouracil dose adjustment based on pharmacokinetic follow-up compared with conventional dosage: Results of a multicenter randomized trial of patients with metastatic colorectal cancer.” J Clin Oncol. 13:2099-2105, 2008.)—though using data from a single sampling and not from PK profiling—our analysis strongly suggests that Abraxane dosing would benefit from therapeutic drug monitoring, especially using PK profile for dose adjustment. It is expected that Genexol-PM and Nanoxel would have the same PK variations. The wide variability of Abraxane was rather surprising. The role of therapeutic drug monitoring is to reduce this variability. Though PK profiles for Abraxane and Taxol were similar, there are differences. These differences, however, are small and could be masked by the large variation in PK of Abraxane/Taxol.

### Example 6

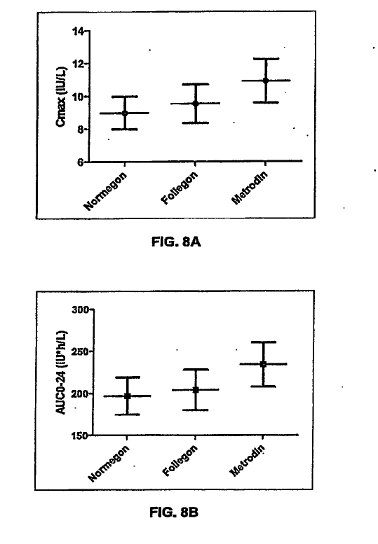

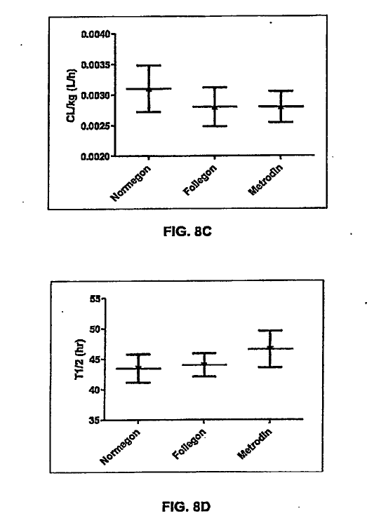

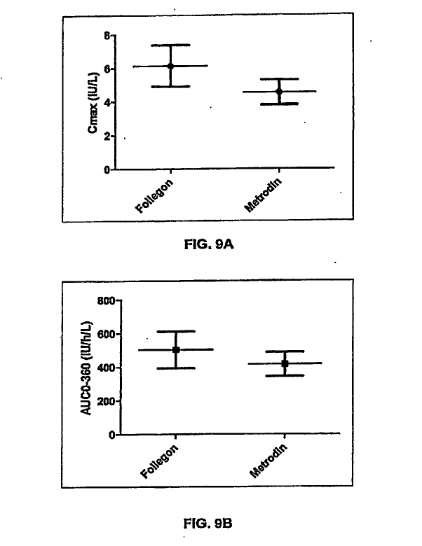

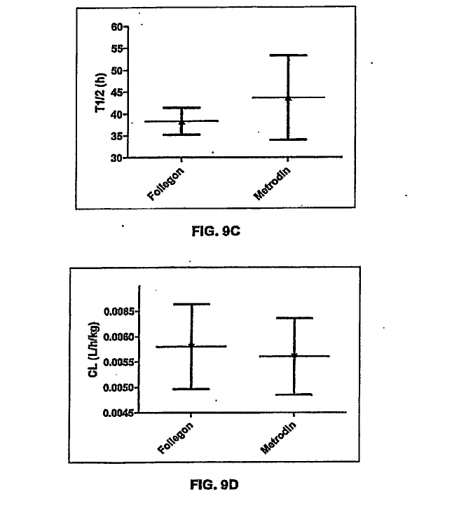

To determine whether medication used in assisted reproduction, i.e. FSH (follicle stimulating hormone), we performed a PK analysis using published literature on FSH PK. Shown below are the plots of FSH PK across several studies (FIGS. 8 and 9) (Duijkers I J, Klipping C, Boerrigter P J, Machielsen C S, De Bie J J, Voortman G. (2002), Single dose pharmacokinetics and effects on follicular growth and serum hormones of a long-acting recombinant FSH preparation (FSH-CTP) in healthy pituitary-suppressed females. Hum Reprod. 17:1987-93.; Out H J, Schnabel P G, Rombout F, Geurts T B, Bosschaert M A, Coelingh Bennink H J. (1996) A bioequivalence study of two urinary follicle stimulating hormone preparations: Follegon and Metrodin. Hum Reprod. 11:61-3.). To compare it to 5-FU data, we used variability as SD/Mean. The AUC variability was calculated at 26.08% and 20.57% for IM Follegon and Metrodin, respectively; 17.72%, 18.42%, 17.69% for sc Normegon, Follegon, and Metrodin, respectively. Surprisingly, this variation is close to the 5-FU variability of 34.42% (Ychou M, Duffour J, Pinguet F, Kramar A, Joulia J M, Topart D, Bressolle F. (1999) Individual 5-FU dose adaptation schedule using bimonthly pharmacokinetically-modulated LV5-FU2 regimen: a feasibility study in patients with advanced colorectal cancer. Anticancer Res. 19:2229-35.) The analysis suggested that therapeutic drug monitoring for FSH could be as beneficial as therapeutic drug monitoring for 5-FU.

### Example 7

### Determination of Minimum Datapoints Necessary for PK Profiling

For the single accessible pool model, following a bolus injection of amount D into the pool, the pharmacokinetic data can be described by a sum of exponentials equation of the general form shown in this equation:

C(t)=C0^{-t/τ1}+ . . . +C0^{-t/τn}

This allows for the quantitation of PK variables once the exponentials been patients with metastatic colorectal cancer.” J Clin Oncol. 13:2099-2105, 2008.)—though using data from a single sampling and not from PK profiling—our analysis strongly suggests that Abraxane dosing would benefit from therapeutic drug monitoring, especially using PK profile for dose adjustment. It is expected that Genexol-PM and Nanoxel would have the same PK variations. The wide variability of Abraxane was rather surprising. The role of therapeutic drug monitoring is to reduce this variability. Though PK profiles for Abraxane and Taxol were similar, there are differences. These differences, however, are small and could be masked by the large variation in PK of Abraxane/Taxol.

### Example 6

To determine whether medication used in assisted reproduction, i.e. FSH (follicle stimulating hormone), we performed a PK analysis using published literature on FSH PK. Shown below are the plots of FSH PK across several studies (FIGS. 8 and 9) (Duijkers I J, Klipping C, Boerrigter P J, Machielsen C S, De Bie J J, Voortman G. (2002), Single dose pharmacokinetics and effects on follicular growth and serum hormones of a long-acting recombinant FSH preparation (FSH-CTP) in healthy pituitary-suppressed females. Hum Reprod. 17:1987-93.; Out H J, Schnabel P G, Rombout F, Geurts T B, Bosschaert M A, Coelingh Bennink H J. (1996) A bioequivalence study of two urinary follicle stimulating hormone preparations: Follegon and Metrodin. Hum Reprod. 11:61-3.). To compare it to 5-FU data, we used variability as SD/Mean. The AUC variability was calculated at 26.08% and 20.57% for IM Follegon and Metrodin, respectively; 17.72%, 18.42%, 17.69% for sc Normegon, Follegon, and Metrodin, respectively. Surprisingly, this variation is close to the 5-FU variability of 34.42% (Ychou M, Duffour J, Pinguet F, Kramar A, Joulia J M, Topart D, Bressolle F. (1999) Individual 5-FU dose adaptation schedule using bimonthly pharmacokinetically-modulated LV5-FU2 regimen: a feasibility study in patients with advanced colorectal cancer. Anticancer Res. 19:2229-35.) The analysis suggested that therapeutic drug monitoring for FSH could be as beneficial as therapeutic drug monitoring for 5-FU.

### Example 7

### Determination of Minimum Datapoints Necessary for PK Profiling

For the single accessible pool model, following a bolus injection of amount D into the pool, the pharmacokinetic data can be described by a sum of exponentials equation of the general form shown in this equation:

C(t)=C0^{-t/τ1}+ . . . +C0^{-t/τn}

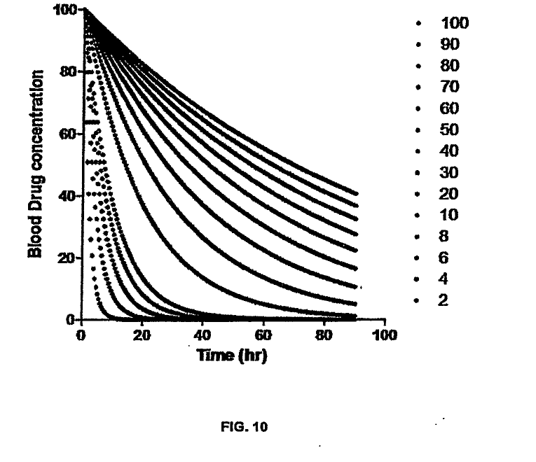

This allows for the quantitation of PK variables once the exponentials been defined. It has to be done with clinical data from a full pharmacokinetic study so that the exponentials (τ) can be defined. The modeling to define the exponentials can be done using any pharmacokinetic programs capable of performing compartmental modeling. They would include programs such as WinNonlin, EquivTest, Kinetica, Phoenix/WinNonlin, and PK Solutions. Once defined, exponentials can be used to generate full PK profile using n+1 samples. As shown below (FIG. 10)—for a single exponential, this would mean two datapoints collected so that they bracket the PK curve-preferably early during the 1st quarter of the descend and a timepoint late during the last quarter of the descend. Usual PK profile would not have at least two exponentials. Each additional exponential requires additional two datapoints. The datapoints will have to be positioned at the first quarter of the descend and last quarter of the descend ending at the intersection of the two components. This is necessary as exponential can only be accurately modeled with two datapoint along its dominant segment. Since the 2nd datapoint of exponential 1 overlap with the 1st datapoint of exponential 2, only 3 datapoints are needed (or n+1). This is shown in FIG. 11. For IV infusion and others modes of administration with an absorption phase, the bioavailability and rate of absorption will have to enter into the equation on top of these exponentials. Those are also regulated by exponentials and can be treated in the same manner.

Typical profile of single exponential PK curve—assuming bolus administration. Two datapoints are sufficient to determine the PK profile for single exponential PK. Starting concentration was placed at 100% and the slow decaying PK with tau of 100 is shown as the top first curve with progressively decreasing tau for subsequent curves. Visualizing the exponential allows for the appropriate placement of the datapoints. The placement of the 1st and 2nd datapoints would be at 75-100 and 0-25 blood drug concentration.

FIG. 11 shows a PK profile characterized by two exponentials. The early part of the curve (distribution phase) is dominated by exponential 1 and the later part of the curve (clearance phase) is dominated by exponential 2. Visualizing the exponential allows for the appropriate placement of the datapoints. The placement of 1st/2nd data point for exponential 1 would be 0-1 hr and 2-4 hr; the placement of 1st/2nd datapoints for exponential 2 would be 2-4 hr and 29-39 hr.

### Example 8

Detection of paclitaxel on a prototypic lateral flow assay for PK profiling. To enable PK profiling, we developed point-of-care assay for various analytes—one of them is paclitaxel. Paclitaxel is routinely detected LC/MS/MS with detection level of 1-5 ng/ml. ELISA using mAb against paclitaxel has also been developed with comparable detection level. These ELISA assays, despite being simpler than the LC/MS/MS method, are still impractical for field application requiring blood drawing, separation of blood into serum, and testing on microtiter plate format in clinical lab by trained personnel. Our innovation is in the development of a lateral flow assay that can accept finger pricked blood and read by a reader that can be deployed at point-of-care (in home, doctor office, or central lab). The ease of sampling and testing is compatible with multiple sampling during the course of treatment so that detail pharmacokinetic data can be obtained.

Test strips was constructed by annealing the cellulose absorbent pad (Cellulose Fiber Sample Pads 17 mm×100 m CFSP001700 (Millipore, Bedford, Mass.)) and glass conjugate fiber (Glass Fiber Conjugate Pads 8 mm×100 m GFCP000800 (Millipore, Bedford, Mass.)) onto the membrane card (Hi-Flow Plus 240 Membrane cards 60 m×301 mm HF240MC100 (Millipore, Bedford, Mass.)). The assembled card was cut with guillotine cutter to yield 4 mm strips. The guillotine cutter available on site is an Index Cutter II from A-Point Technologies Inc., Gibbstown, N.J., USA.

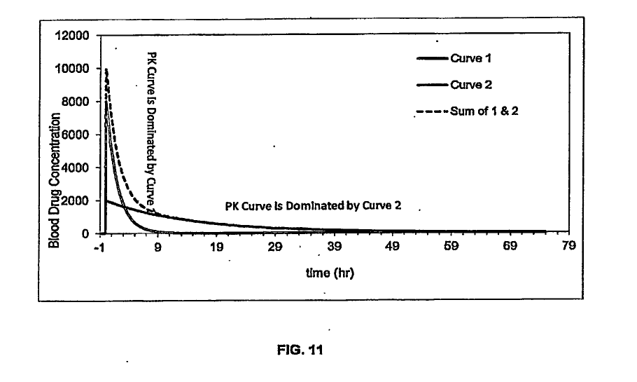

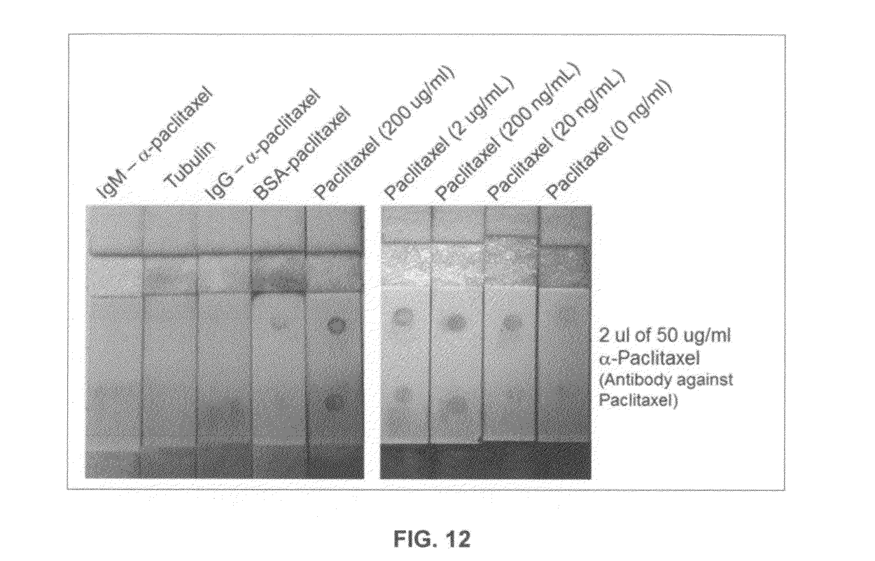

Initial testing of paclitaxel-BSA at 20:1 ratio and mAb colloidal conjugation at 1 mg/ml at pl of each mAb was performed to determine positive binding. The experiment shown in FIG. 12 indicates that paclitaxel-BSA/anti-paclitaxel mAb-colloidal gold conjugate pair generated sufficient signal. Signal was much stronger, however, with BSA-paclitaxel/anti-paclitaxel mAb-colloidal gold pair when the mAb was spotted onto the membrane and BSA-paclitaxel flowed through the membrane (left panel, FIG. 12). As shown (right panel, FIG. 12), a much stronger signal was detected when anti-paclitaxel mAb was immobilized on the nitrocellulose membrane and paclitaxel-gold conjugate flowed through the membrane. The paclitaxel was mixed with serum and the mixture labeled directly with nanogold particles. Detection limit down to 20 ng/ml of paclitaxel was possible.

### Example 9

Quantitative lateral flow assay using Qiagen lateral flow reader. This example shows how lateral flow cassettes can be converted to quantitative point-of-care device using appropriate readers such as those produced by Qiagen.

Quantitative point-of-care lateral flow assay for hCG was constructed using conventional hCG lateral flow cassettes with quantitation using Qiagen lateral flow reader configured so that the calibration curve is embedded into a 2D bar code imprinted the cassette allowing all the quantitative process be uploaded onto the reader in the field and quantitation occur in the background without need for expert technical support at point-of-care/point of use.

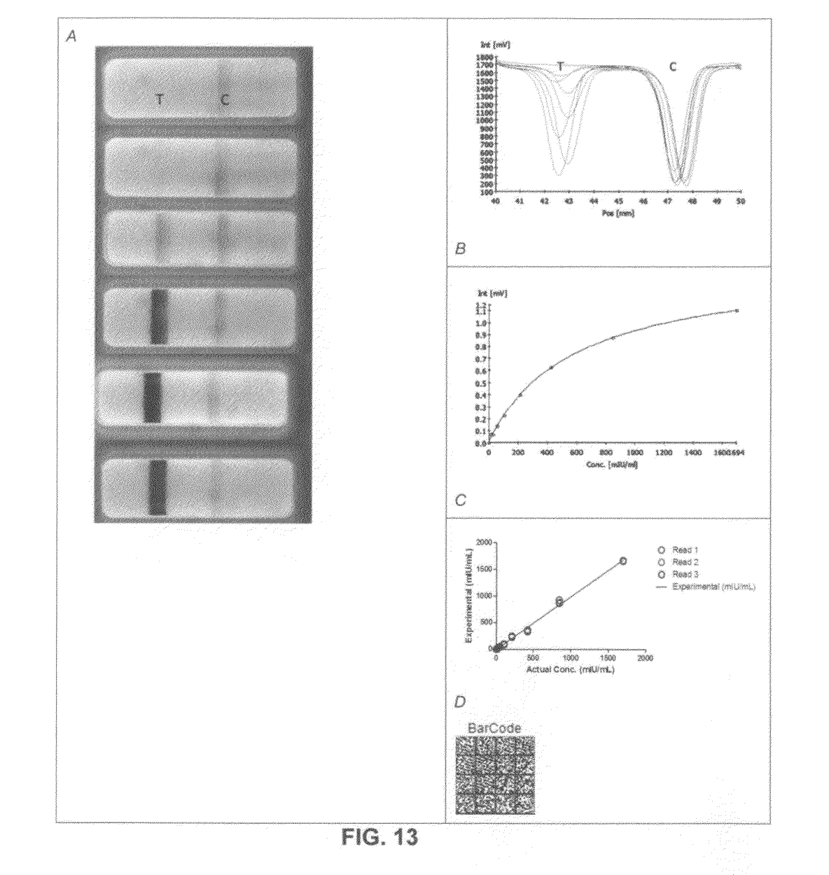

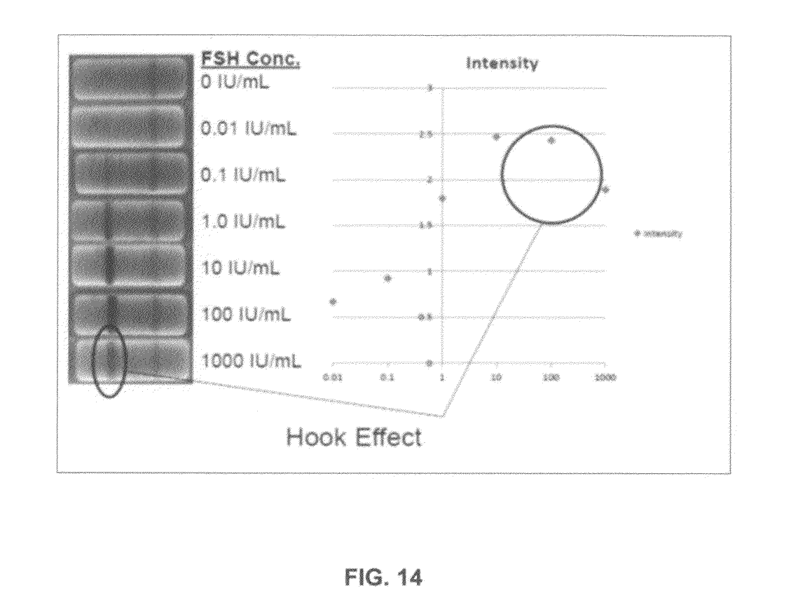

FIG. 13 shows the results of quantitative lateral flow TDM for hCG, hCG a hormone commonly used to induce ovulation in assisted reproduction. Sera spiked with increasing levels of hCG (top to bottom=0-1694 mIU/mL) were tested by a quantitative lateral flow device for hCG (FIG. 13A). The device was scanned by a reader and the scans are shown in FIG. 13B. The ratio of peak areas for T and C were plotted versus concentration to establish the calibration curve (FIG. 13C). The calibrate device was used to quantitate unknown hCG levels, with excellent agreement between expected and experimental (FIG. 13D). All of these data are saved as two dimension bar code which can be read by any of the readers to establish the parameters for analysis of unknown samples.

### Example 10

Quantitative lateral flow assay using image analyzer ImagePro program coupled to image capture by Olympus SXX16 microscope equipped with Evolution MP camera. This example demonstrates the use of any image analysis program such as ImagePro and any camera systems including mobile phone camera. As long as the image of sufficient quality for the comparison of intensity of the test line versus the control line, it is possible to use it for quantitation which could happen on site or remotely. Lateral cassettes for FSH was developed with increasing concentration of FSH and image captured analyzed by ImagePro and the Intensity ratio of test over control was used to plot intensity vs. concentration showing good quantitative trend. The assay in its current format suffered from the hook effect—reduction in signal at extreme levels of FSH. The hook effect is quite important and means for avoiding it are being developed.

### Example 11

Stabilization of lateral flow device signal for quantitation at a later date either at point of use/point-of-care or at centralized lab since the stability of the signal allows for the shipment of the devices back to the centralized lab after development at point-of-care.

Quantitation was performed at completion of flow (i.e., 15-20 minutes) and monitored out to 72 hours to insure the stability of the signal. Signal stability is necessary in order for the cassette to be shipped back to central testing labs. As shown below, all quantitative tested developed by us to date exhibited stability of at least 72 hr. In most cases, stability was in weeks and months. Stability was insured using the appropriate configuration of the cassettes, buffer condition, and sample volume size. Once the signal been stabilized, we found that it will remained stable at least several months out.

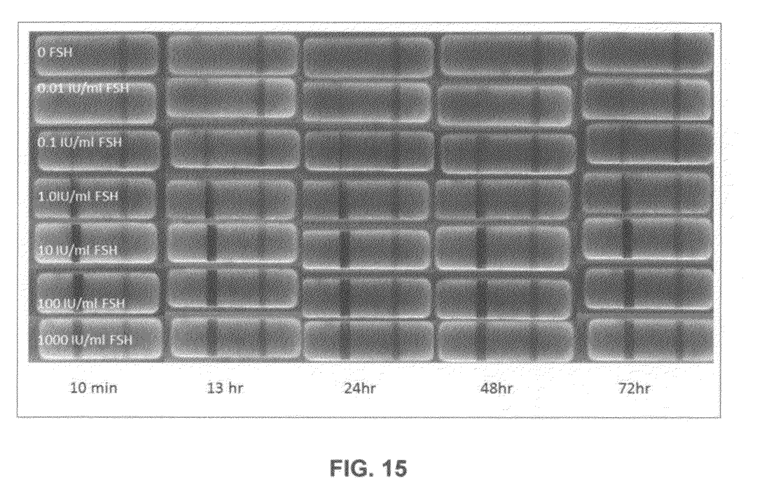

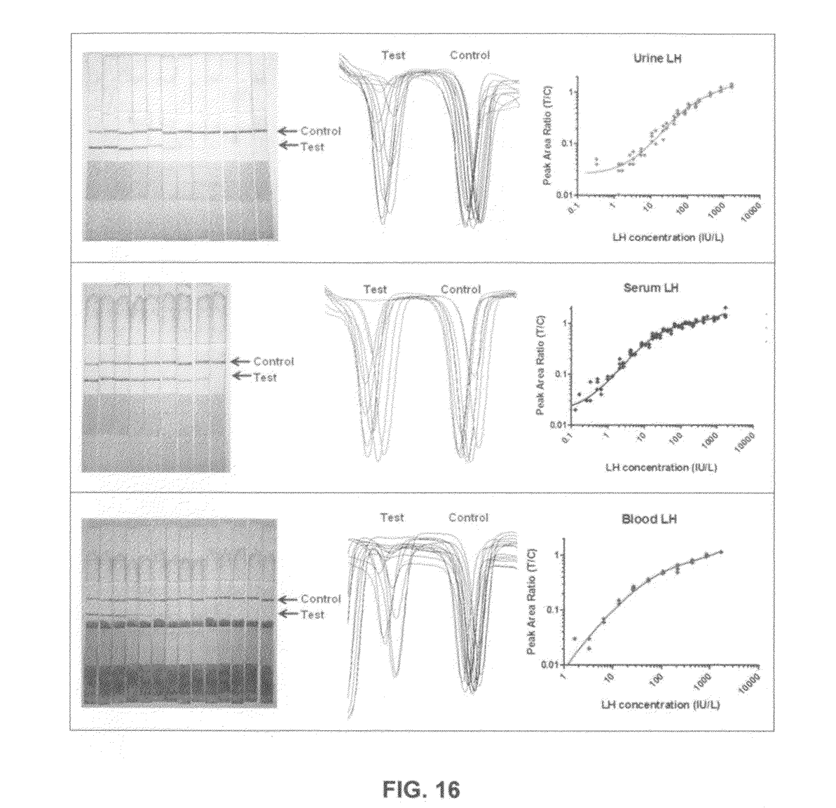

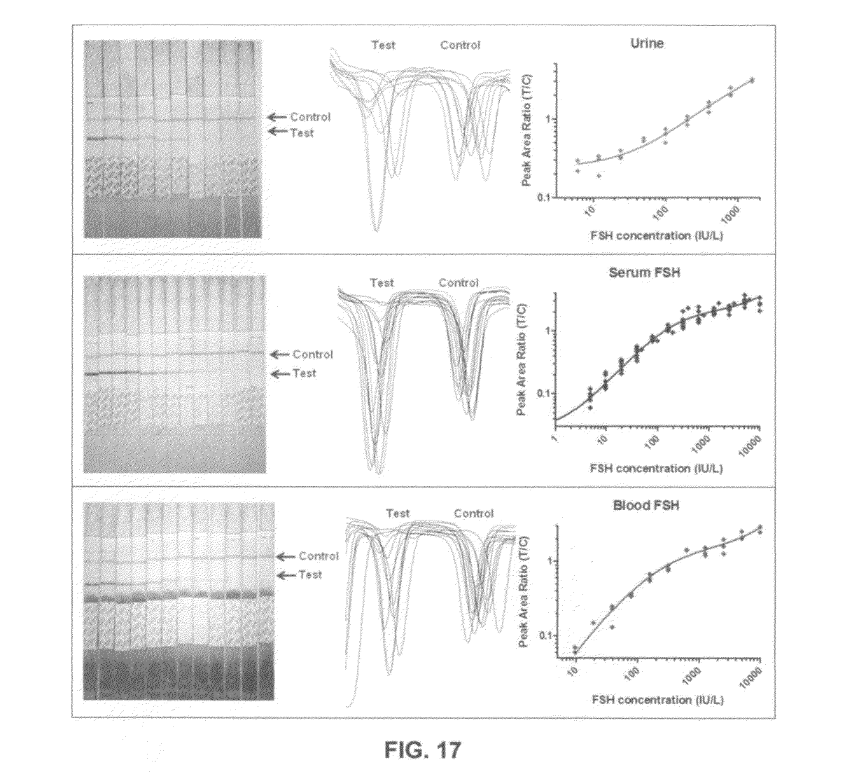

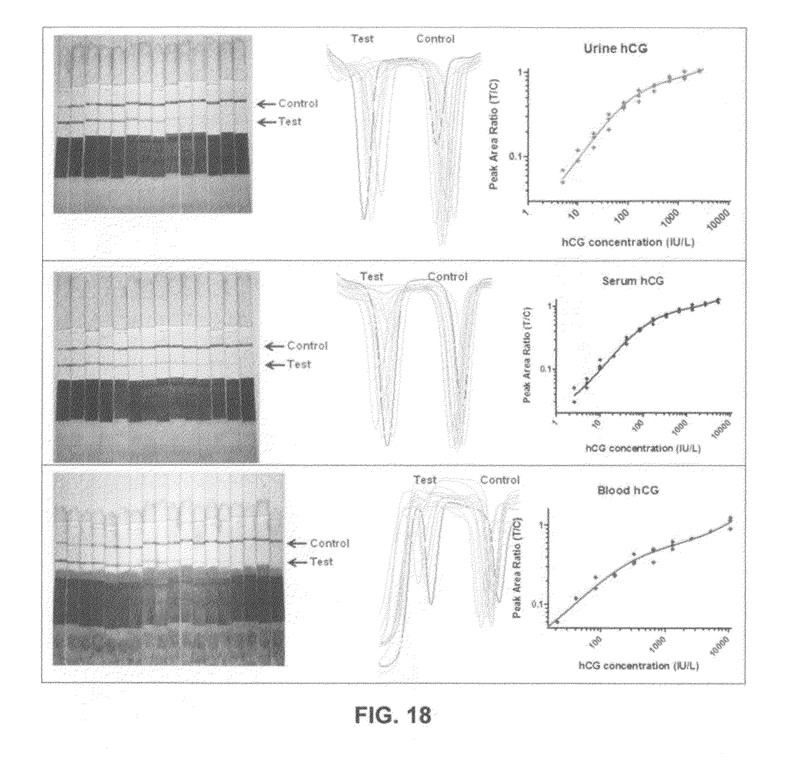

FIG. 15 illustrates the stability of the signal for an FSH quantitative nanogold lateral

### Example 12

Construction of quantitative point-of-care assay for hCG, LH, and FSH. IVF has a delivery rate per started cycle of around 22% and close to 250,000 children where 26% of pregnancies were twins and 2.5% triplets. IVF Protocols—with a target of generating between 8 and 15 oocytes—are complex, time consuming, expensive and may give rise to considerable patient discomfort and increased chance of complications, especially the ovarian hyperstimulation syndrome (OHSS). The drug regimen used most often for ovarian hyperstimulation consists of a steroid contraceptive pretreatment to regulate the menstrual cycle, GnRH agonist analogues to suppress LH release in the pre-stimulation cycle, daily gonadotropin injections for almost 2 weeks to promote the development of multiple egg follicles, as well as a bolus dose of hCG to induce ovulation of the mature oocytes. In many countries, medication and monitoring expenses outweigh the cost of the IVF procedure itself. Therefore, there is a shift in emphasis from mild stimulation towards mild ovarian response is ongoing to reduce both cancellation and over-response rates by developing more individualized treatment regimens based on initial patient characteristics, such as age, body weight and ovarian reserve characteristics. To facilitate individualized treatment regimens that would reduce the cost of medication and OHSS risk due to overmedication, we have developed point-of-care therapeutic drug monitoring of the gonadotropins widely used in IVF—FSH, LH, and hCG.

The lateral flow assays were constructed using mAb pairs against LH, FSH and hCG. The reflectometric optical reader, which utilizes confocal optics with a low distance to target ratio, was used. Serum, Urine from pre-puberty females, and Blood from female donors were used as matrices to spike the standards and thus generate samples for these experiments. Volume used was 40 uL per cassette. For hCG blood assay, the volume was reduced to 30 ul per cassette. The following standards were used: LH reference standard from Genway, San Diego, Calif. (lot#A11072104), FSH reference standard from Genway, San Diego, Calif. (lot#11-663-45941), and hCG reference standard from AbdSerotec, Raleigh, N.C. (lot#120811). The data were plotted and analyzed using GraphPad Prism, San Diego, Calif.

The described embodiments are to be considered in all respects only as illustrative and not restrictive, and the scope of the present invention is not limited to the foregoing description. Those of skill in the art may recognize changes, substitutions, adaptations and other modifications that may nonetheless come within the scope of the present invention and range of the present invention.

1. A method for therapeutic drug monitoring of an individual treated with a drug using pharmacokinetic profiling, the method comprising:
constructing a pharmacokinetic profile of the drug for the individual using concentrations of drug in at least two samples obtained from the individual at time points suitable to construct a pharmacokinetic profile, the at least two samples collected at point-of-care or point of use by sampling or self-sampling on point-of-care devices or point of use devices, each capable of quantitating the drug, or on matrices suitable for storage of the at least two samples prior to quantitation of the drug by a laboratory, the pharmacokinetic profile including one or more pharmacokinetic parameters suitable for guiding dosing of the drug for the individual.

2. The method of claim 1, wherein at least two samples includes from 2-12 samples.

3. The method of claim 2, wherein at least two samples are collected over a time period of up to 8 hours, up to 24 hours, up to 48 hours, or up to 72 hours.

4. The method of claim 2, wherein the pharmacokinetic parameters are selected from the group consisting of AUC, AUC_{inf}, Tₘₐₓ, Cₘₐₓ, time above threshold, steady state concentration, absorption rate, clearance rate, distribution rate, terminal T_{1/2} or parameters drawn from noncompartmental pharmacokinetic or compartmental pharmacokinetic analysis, including physiological model-based compartmental pharmacokinetic analysis.

5. The method of claim 4, further comprising generating a report including the pharmacokinetic profile of the individual.

6. The method of claim 5, wherein the report includes a recommendation regarding dosing based on the pharmacokinetic profile of the individual.

7. The method of claim 6, wherein a reduction in drug dosage is indicated to reduce risk of toxicity based on one or more pharmacokinetic parameters.

8. The method of claim 7, wherein the reduction in drug dosage is indicated based on time above threshold, wherein the threshold is the drug concentration above which toxicity occurs, or one or more of AUC, AUCinf, MRT, exponentials defining the pharmacokinetic profile, Vss, Vz or combination of a group of pharmacokinetic variable to adequately describe the pharmacokinetic profile.

9. The method of claim 6, wherein a drug dose adjustment is indicated to increase efficacy based on one or more pharmacokinetic parameters.

10. The method of claim 9, wherein an increase in drug dosage is indicated based on one or more of AUC, AUCinf, MRT, exponentials defining the pharmacokinetic profile, Vss, Vz or combination of a group of pharmacokinetic variables to adequately describe the pharmacokinetic profile.

11. The method of claim 10, wherein the dose is adjusted to within 5-25% of a desired target value.

12. The method of claim 1, wherein each of the at least two samples is applied to the point-of-care device or the point of use device for determining the concentration of the drug, wherein the point-of-care device or the point of use device comprises a lateral flow strip having a construction and composition such that an application of one or more of the at least two samples to the lateral flow strip causes a fraction of the drug in the sample to bind to with a component of the lateral flow strip such that a detectable signal proportional to the concentration of the drug in the applied sample is produced.

13. (canceled)

14. (canceled)

15. (canceled)

16. The method of claim 1, wherein the drug is a chemotherapeutic agent.

17. (canceled)

18. The method of claim 16, wherein the drug is a taxane.

19. The method of claim 18, wherein the drug is a paclitaxel formulation.

20. The method of claim 19, wherein the paclitaxel formulation is selected from the group consisting of albumin bound paclitaxel, polymeric micelle paclitaxel, polymeric nanoparticle formulation of paclitaxel, vitamin E-based paclitaxel emulsion, polyglutamate paclitaxel.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The method of claim 12, wherein the drug in the sample competes with a detectably labeled molecule in the lateral flow strip for binding to an antibody or ligand.

33. The method of claim 12, wherein the drug competes with a molecule immobilized in a defined region of the lateral flow strip for binding to a detectably labeled antibody or ligand.

34. The method of claim 12, further comprising detecting the detectable signal of the lateral flow strip and correlating the signal with the concentration of the drug in the sample.

35. The method of claim 12, wherein the sample is whole blood.

36. The method of claim 12, wherein the lateral flow strip comprises two or more distinct antibodies having different affinities for the drug.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. A method for therapeutic drug monitoring of an individual treated with a drug using pharmacokinetic profiling, the method comprising:
constructing a pharmacokinetic profile of the drug for the individual using concentrations of drug in at least two samples obtained from the individual at time points suitable to construct a pharmacokinetic profile, the at least two samples collected at point of use by self-sampling on point of use devices capable of quantitating the drug, the pharmacokinetic profile including one or more pharmacokinetic parameter(s) suitable for guiding dosing of the drug for the individual.

52. The method of claim 51, wherein at least two samples is 4 samples.

53. The method of claim 52, wherein at least two samples are collected over a time period of up 24 hours.

54. The method of claim 53, wherein the pharmacokinetic parameter is area under the curve (AUC).

55. The method of claim 53, wherein the pharmacokinetic parameter is mean residence time (MRT).

56. The method of claim 53, wherein the pharmacokinetic parameters include AUC, AUC_{inf}, Tₘₐₓ, Cₘₐₓ, time above threshold, steady state concentration, absorption rate, clearance rate, distribution rate, and terminal T_{1/2}.

56. A method for constructing a pharmacokinetic profile of a patient comprising:
(a) treating the patient with a drug;
(b) collecting a first blood sample from a patient by self-sampling within two (2) hours of the drug treatment;
(c) applying the first blood sample to a point-of-use device;
(d) determining a first set of pharmacokinetic parameters for the drug in the patient;
(e) collecting a second blood sample from a patient by self-sampling within twelve (12) hours of the drug treatment and after the collection of the first blood sample;
(f) applying the second blood sample to a point-of-use device;
(g) determining a second set of pharmacokinetic parameters for the drug in the patient; and
(h) comparing the first set of pharmacokinetic parameters with the second set of pharmacokinetic parameters to obtain a pharmacokinetic profile.

57. The method of claim 56, wherein the pharmacokinetic parameters include AUC, AUC_{inf}, Tₘₐₓ, Cₘₐₓ, time above threshold, steady state concentration, absorption rate, clearance rate, distribution rate, and terminal T_{1/2}.

58. The method of claim 57 wherein the point-of-care device is a lateral flow device.

59. A method for adjusting the dosage of a drug in a patient being treated with the drug comprising:
(a) treating the patient with a drug;
(b) collecting a first blood sample from a patient by self-sampling within two (2) hours of the drug treatment;
(c) applying the first blood sample to a point-of-use device;
(d) determining a first set of pharmacokinetic parameters for the drug in the patient;
(e) collecting a second blood sample from a patient by self-sampling within twelve (12) hours of the drug treatment and after the collection of the first blood sample;
(f) applying the second blood sample to a point-of-use device;
(g) determining a second set of pharmacokinetic parameters for the drug in the patient; and
(h) comparing the first set of pharmacokinetic parameters with the second set of pharmacokinetic parameters to obtain a pharmacokinetic profile for the patient.
(i) comparing the pharmacokinetic profile of the patient with an idealized pharmacokinetic profile for the drug; and
(j) adjusting the dosage of the drug for treating the patient based on the comparison between the pharmacokinetic profile of the patient with an idealized pharmacokinetic profile for the drug.

60. The method of claim 59, wherein the pharmacokinetic parameters include AUC, AUC_{inf}, Tₘₐₓ, Cₘₐₓ, time above threshold, steady state concentration, absorption rate, clearance rate, distribution rate, and terminal T_{1/2}.

61. The method of claim 60 wherein the point-of-care device is a lateral flow device.
